(19) United States
(12) Patent Application Publication
(10) Pub. No.: US 2017/0247346 A1
(43) Pub. Date: Aug. 31, 2017

(54) TETRAHYDROFURAN DERIVATIVES

(71) Applicant: BASF SE, Ludwigshafen (DE)

(72) Inventors: Christina Haaf-Kleinhubbert, Hemsbach (DE); Michele Gatti, Frick (SE); Gijsbrecht Jacobus Maria Habraken, Budd Lake, NJ (US)

(21) Appl. No.: 15/519,850

(22) PCT Filed: Oct. 9, 2015
(86) PCT No.: PCT/EP2015/073364

§ 371 Date: Apr. 18, 2017

(30) Foreign Application Priority Data
Oct. 20, 2014 (EP) 14189499.8

(51) Int. Cl. C07D 307/12; B05D 3/06; B05D 7/02; C09D 4/00

(52) U.S. Cl. CPC C07D 307/12; C09D 4/00; B05D 3/067; B05D 7/02

(57) ABSTRACT

The invention relates to tetrahydrofuran derivatives of the formula (I)

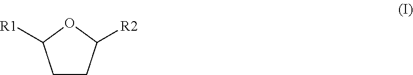

in which the radical R1 has the definition (CH₂═CH—CO—O—(CHR3—CH₂—O)ₘ—CH₂)— and the radical R2 has the definition (CH₂═CH—CO—O—(CHR4—CH₂—O)ₙ—CH₂)—, in which the radicals R3 and R4 independently of one another are hydrogen or methyl, and with the proviso that the sum of the indices m and n is a number in the range from 0 to 20.

The compounds (I) are suitable for coating the surfaces of solid substrates, more particularly for coating plastics.

### FIELD OF THE INVENTION

The present invention relates to tetrahydrofuran derivatives of specific structure which are suitable for coating the surfaces of solid substrates, especially of plastics.

### PRIOR ART

EP-A-043,448 describes dimethacrylic esters of 2,5-dimethyloltetrahydrofuran. According to the disclosure at page 4, lines 13-14, these compounds are substances of high viscosity. The compounds find use as an essential constituent of sealants and/or adhesives which harden in the absence of oxygen.

DE-A-10,2010,044,206 describes a method for producing radiation-curable (meth)acrylates on the basis of propoxylated glycerol. First of all (meth)acrylic acid is reacted with tri- to tetra-propoxylated glycerol, followed by removal of excess (meth)acrylic acid from the resulting reaction mixture by aqueous extraction.

DE-A-10,2010,044,206 describes a method for producing radiation-curable (meth)acrylates on the basis of propoxylated glycerol. First of all (meth)acrylic acid is reacted with glycerol having 2.9- to 4-fold ethoxylation, followed by removal of excess (meth)acrylic acid from the resulting reaction mixture by reaction with at least one aromatic or aliphatic epoxide with a functionality of at least two.

### DESCRIPTION OF THE INVENTION

It was an object of the present invention to provide substances suitable for coating the surfaces of solid substrates, and especially plastics, where the coating is cured by radiation curing, more particularly with UV light. The cured coatings, especially the UV-cured coating materials, ought to have high hardness.

The substances for development ought in particular to meet all of the following three criteria:

(1) The viscosity of the substances as such ought to be below 500 mPas (measured with a Brookfield viscometer at 25° C., shear rate of 1000 s-1, in accordance with DIN EN ISO 3219/A.3).

(2) The pendulum hardness of the coatings resulting when the substances have been applied to the surfaces of solid substrates and cured using UV radiation is to be higher than 70 sec (König pendulum hardness, measured according to DIN 53157; with this method, the figure reported is the pendulum damping in seconds).

(3) The adhesion to plastic is to be 0 to 2, preferably 0 or 1 (measured by the DIN EN ISO 2409 cross-cut method, with the G values being situated according to the school-grade system in the range from 0 to 5, where 0 is the best and 5 the worst score).

The invention provides first of all tetrahydrofuran derivatives of the formula (I)

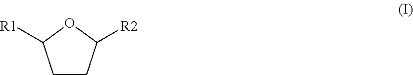

in which the radical R1 has the definition (CH₂═CH—CO—O—(CHR3—CH₂—O)ₘ—CH₂)— and the radical R2 has the definition (CH₂═CH—CO—O—(CHR4—CH₂—O)ₙ—CH₂)—, in which the radicals R3 and R4 independently of one another are hydrogen or methyl, and with the proviso that the sum of the indices m and n is a number in the range from 0 to 20.

Accordingly, an equivalent notation for the compounds (I) is the following structure (A):

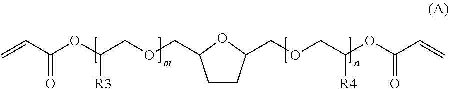

In light of formula (I), the substituents R1 and R2 may be located either on the same side or on different sides of the reference plane dictated by the five-membered ring. In other words, the compounds (I) may be present in the cis or the trans form. These isomers would be denoted accordingly as cis-2,5-R1-R2-tetrahydrofuran and trans-2,5-R1-R2-tetrahydrofuran, respectively.

In one embodiment the sum of the indices m and n is a number in the range from 4 to 15 and more particularly from 5 to 12.

The compounds (I) may be prepared per se by all of the methods known to the chemist. Preferably, the compounds (I) are prepared as follows: 2.5-dimethyloltetrahydrofuran, which has the formula (B),

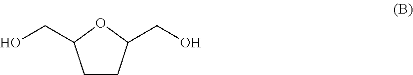

is reacted, either directly or after having been ethoxylated and/or propoxylated beforehand, with acrylic acid. Operation here takes place preferably with an excess of acrylic acid in an organic solvent, especially cyclohexane or methylcyclohexane, and in the presence of an acid esterification catalyst, more particularly methanesulfonic acid, sulfuric acid or p-toluene-sulfonic acid.

Preference is given to using technical mixtures of the diol (B), with the (molar) cis/trans ratio being situated in particular in the range of 95:5 and 5:95. Particularly preferred here is a (molar) cis/trans ratio in the range of 95:5 and 50:50 and particularly in the range of 95:5 and 80:20.

After the end of the esterification, solvent used is removed, preferably by distillation, especially under reduced pressure. Also removed is excess acrylic acid, which can be done in a variety of ways, such as by distillation, by extractive washing or by chemical means.

If excess acrylic acid is removed by extractive washing, the following applies: washing is carried out using preferably an extraction with aqueous medium (in this regard compare, for example, the relevant disclosure content of DE-A-102010044206).

If excess acrylic acid is removed chemically by scavenging, the following applies: the chemical scavenging is accomplished preferably by reaction of the excess acrylic acid with epoxide compounds, more particularly epoxide compounds having a functionality of at least two (in this regard compare, for example, the relevant disclosure content in DE-A-10,2010,044204).

A further subject of the invention is coating compositions comprising one or more tetrahydrofuran derivatives of formula (I)

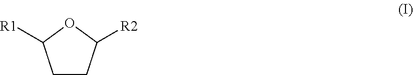

in which the radical R1 has the definition (CH₂═CH—CO—O—(CHR3—CH₂—O)ₘ—CH₂)— and the radical R2 has the definition (CH₂═CH—CO—O—(CHR4—CH₂—O)ₙ—CH₂)—, in which the radicals R3 and R4 independently of one another are hydrogen or methyl, and with the proviso that the sum of the indices m and n is a number in the range from 0 to 20.

In one embodiment the sum of the indices m and n is a number in the range from 4 to 15 and more particularly from 5 to 12.

With regard to the preparation of the compounds (I), reference may be made to the statements above, in terms both of the general remarks and of the preferred embodiments.

If—as observed above—excess acrylic acid is removed chemically by scavenging, the following applies: the chemical scavenging is accomplished preferably by reaction of the excess acrylic acid with epoxide compounds, more particularly epoxide compounds having a functionality of at least two (in this regard compare, for example, the relevant disclosure content in DE-A-10,2010,044204). The result is a composition which comprises the compounds (I) and also the resultant scavenging products.

### Use of the Compositions

A further subject of the invention is the use of the compounds (I) for coating the surfaces of solid substrates. There is no restriction here on the nature of the substrate as such.

Examples of suitable substrates are, for example, textile, leather, metal, plastic, glass, wood, paper or cardboard.

In one particularly preferred embodiment the substrates in question are plastics.

Plastics—in line with the usual linguistic usage—and for the purposes of the present specification are organic, polymeric solids. Plastics are typically divided into three major groups: thermoplastics, thermosets, and elastomers. In the general language, “plastic” is the generic term. Plastics are conventionally produced synthetically or semisynthetically from monomeric organic molecules or biopolymers.

Examples of suitable substrates for the coating compositions of the invention are thermoplastic polymers, especially polymethyl methacrylates, polybutyl methacrylates, polyethylene terephthalates, polybutylene terephthalates, polyvinylidene fluorides, polyvinyl chlorides, polyesters, polyolefins, acrylonitrile-ethylene-propylene-diene-stryene copolymers (A—EPDM), polyetherimides, polyetherketones, polyphenylene sulfides, polyphenylene ethers or mixtures thereof.

Mention may further be made of polyethylene, polypropylene, polystyrene, polybutadiene, polyesters, polyamides, polyethers, polycarbonate, polyvinylacetal, polyacrylonitrile, polyacetal, polyvinyl alcohol, polyvinyl acetate, phenolic resins, urea resins, melamine resins, alkyd resins, epoxy resins or polyurethanes, block copolymers or graft copolymer thereof, and blends of these.

The following plastics may be mentioned as plastics of preferential suitability: acrylonitrile-butadiene-styrene (ABS), polyacrylonitrile/methyl methacrylate (AMMA), acrylonitrile-styrene-acrylate (ASA), epoxy resins (EP), expanded polystyrene (EPS), ethylene-vinyl acetate copolymer (EVA), high-density polyethylene (HDPE), low-density polyethylene (LDPE), methyl methacrylate/acrylonitrile/butadiene/styrene (MABS), methyl acrylate/butadiene/styrene copolymer (MBS), melamine-formaldehyde resin (MF), polyamide (PA), nylon (PA6), nylon (PA66), polyacrylonitrile (PAN), 1,2-polybutadiene (PB), polybutylene terephthalate (PBT), polycarbonate (PC), polyethylene (PE), chlorinated polyethylene (PEC), polyetheretherketone (PEEK), polyetherimide (PEI), polyetherketone (PEK), polyarylethersulfone (PES), polyethylene terephthalate (PET), phenol-formaldehyde resin (PF), polyimide (PI), polyisobutylene (PIB), polymethyl methacrylate (PMMA), polyoxymethylene (POM), polypropylene (PP), polyethylene sulfide (PPS), polystyrene (PS), polysulfone (PSU), polyurethane (PUR), polyvinyl acetate (PVAC), polyvinyl alcohol (PVAL), polyvinyl chloride (PVC), polyvinylidene chloride (PVDC), styrene-acrylonitrile (SAN), styrene-butadiene (SB), urea-formaldehyde resin (UF), unsaturated polyester resin UP plastics (short codes as per DIN 7728) and aliphatic polyketones.

Particularly preferred substrates are polyolefins, such as e.g. PP (polypropylene), which alternatively may be isotactic, syndiotactic or atactic and alternatively unoriented or oriented by uniaxial or biaxial stretching, SAN (styrene-acrylonitrile copolymers), PC (polycarbonates), PVC (polyvinyl chlorides), PMMA (polymethyl methacrylates), PBT poly(butylene terephthalates), PA (polyamides), ASA (acrylonitrile-styrene-acrylate copolymers) and ABS (acrylonitrile-butadiene-styrene copolymers), and also their physical mixtures (blends). Particularly preferred are PP, SAN, ABS, ASA and also blends of ABS or ASA with PA or PBT or PC.

A further subject of the invention is the use of coating compositions comprising one or more compounds (I) for coating the surfaces of solid substrates. There is no restriction here on the nature of the substrate as such. In one particularly preferred embodiment the substrates in question are plastics, which are subject to the comments above.

The term “coating compositions” embraces any kind of compositions applied to the surface of a substrate to be coated and subsequently cured, optionally after drying beforehand. In particular, the term “coating compositions” includes all kinds of surface coating.

As the skilled person is aware, a “surface coating” refers to a coating composition which may be liquid or else pulverulent and which is applied in a thin layer, thinly, to an article, in other words the substrate to be coated, and then is cured. In this regard, see also the section below regarding the term “coating”.

Besides the compounds (I), the coating compositions of the invention may further comprise other, typical coatings additives, examples being antioxidants, stabilizers, activators (accelerators), fillers, pigments, dyes, antistatic agents, flame retardants, thickeners, thixotropic agents, surface-active agents, viscosity modifiers, plasticizers or complexing agents. Furthermore, besides the compounds (I), the coating compositions of the invention may also comprise other radiation-curable components not encompassed by the formula (I).

Thickeners contemplated, in addition to radically (co)polymerizied (co)polymers, are customary organic and inorganic thickeners such as hydroxymethylcellulose or bentonite.

Complexing agents which can be used include, for example, ethylenediamineacetic acid and the salts thereof, and also β-diketones.

Suitable fillers include silicates, examples being silicates obtainable by hydrolysis of silicon tetrachloride, such as Aerosil® from Degussa, siliceous earth, talc, aluminum silicates, magnesium silicates, calcium carbonates, etc.

Suitable stabilizers include typical UV absorbers such as oxanilides, triazines and benzotriazole (the latter available as Tinuvin® brands from Ciba-Spezialitatenchemie), and benzophenones. They can be used alone or together with suitable radical scavengers, examples being sterically hindered amines such as 2,2,6,6-tetramethylpiperidine, 2,6-di-tertbutylpiperidine or derivatives thereof, e.g., bis(2,2,6,6-tetramethyl-4-piperidyl) sebacate. Stabilizers are used customarily in amounts of 0.1 to 5.0% by weight, based on the solid components present in the preparation.

Pigments may likewise be included in the coating compositions. Pigments, according to CD Römpp Chemie Lexikon—Version 1.0, Stuttgart/New York: Georg Thieme Verlag 1995, with reference to DIN 55943, are particulate “chromatic or achromatic colorants, organic or inorganic, which are virtually insoluble in the application media”. “Virtually insoluble” here means a solubility at 25° C. of below 1 g/1000 g of application medium, preferably below 0.5, more preferably below 0.25, very preferably below 0.1, and more particularly below 0.05 g/1000 g of application medium.

If a pigment is used, it should be ensured either that curing is carried out with electron beams or that a photoinitiator is used which, in spite of the pigmentation, can be activated by the radiation introduced—for example, by the photoinitiator exhibiting significant absorbence in a wavelength range in which the pigment is sufficiently transparent to the radiation introduced.

In one preferred embodiment of the present invention, no pigment is used and the coating composition is employed in transparent varnishes/clearcoats.

Examples of pigments include any desired systems of absorption pigments and/or effect pigments, preferably absorption pigments. There are no restrictions at all on the number and selection of the pigment components. They may be adapted as desired to the particular requirements, such as to the desired perceived color, for example.

Effect pigments are all pigments which exhibit a plateletlike structure and which give a surface coating specific decorative color effects. The effect pigments are, for example, all effect-imparting pigments which can be used customarily in automotive finishing and industrial finishing. Examples of such effect pigments are pure metal pigments, such as aluminum, iron or copper pigments, for example; interference pigments, such as titanium dioxide-coated mica, iron oxide-coated mica, mixed oxide-coated mica (e.g., with titanium dioxide and Fe₂O₃ or titanium dioxide and Cr₂O₃), metal oxide-coated aluminum, or liquid-crystal pigments, for example.

The color-imparting absorption pigments are, for example, organic or inorganic absorption pigments which are customary and can be used in the coatings industry. Examples of organic absorption pigments are azo pigments, and phthalocyanine, quinacridone and pyrrolopyrrole pigments. Examples of inorganic absorption pigments are iron oxide pigments, titanium dioxide and carbon black.

Where curing of the coating compositions takes place not with electron beams but instead using UV radiation, there is preferably at least one photoinitiator present that is able to initiate the polymerization of ethylenically unsaturated double bonds (C═C double bonds).

Very generally, it is possible to employ all of the photoinitiators that are relevantly known to the skilled person, of the kind described, for example, in relevant technical publications and monographs.

Those contemplated include, for example:
mono- or bisacylphosphine oxides, for instance 2,4,6-trimethylbenzoyldiphenylphosphine oxide (Lucirin® TPO from BASF SE), ethyl 2,4,6-trimethylbenzoylphenylphosphinate (Lucirin® TPO L from BASF SE), bis(2,4,6-trimethylbenzoyl)phenylphosphine oxide (Irgacure® 819 from Ciba Spezialitätenchemie),
benzophenones, hydroxyacetophenones, phenylglyoxylic acid and its derivatives, or mixtures of these photoinitiators. Examples include the following: benzophenone, acetophenone, acetonaphthoquinone, methyl ethyl ketone, valerophenone, hexanophenone, α-phenylbutyrophenone, p-morpholinopropiophenone, dibenzosuberone, 4-morpholino-benzophenone, 4-morpholinodeoxybenzoin, p-diacetylbenzene, 4-aminobenzophenone, 4′-methoxyacetophenone, β-methylanthraquinone, tert-butylanthraquinone, anthraquinonecarboxylic esters, benzaldehyde, α-tetralone, 9-acetylphenanthrene, 2-acetylphenanthrene, 10-thioxanthenone, 3-acetylphenanthrene, 3 -acetylindole, 9-fluorenone, 1-indanone, 1,3,4-triacetylbenzene, thioxanthen-9-one, xanthen-9-one, 2,4-dimethylthioxanthone, 2,4-diethylthioxanthone, 2,4-diisopropylthioxanthone, 2,4-dichlorothioxanthone, benzoin, benzoin isobutyl ether, chloroxanthenone, benzoin tetrahydropyranyl ether, benzoin methyl ether, benzoin ethyl ether, benzoin butyl ether, benzoin isopropyl ether, 7H-benzoin methyl ether, benz[de]anthracen-7-one, 1-naphthaldehyde, 4,4′-bis(dimethylamino)benzophenone, 4-phenylbenzophenone, 4-chlorobenzophenone, Michler's ketone, 1-acetonaphthone, 2-acetonaphthone, 1-benzoylcyclohexan-1-ol, 2-hydroxy-2,2-dimethylacetophenone, 2,2-dimethoxy-2-phenylacetophenone, 2,2-diethoxy-2-phenylacetophenone, 1,1-dichloroacetophenone, 1-hydroxyacetophenone, acetophenone dimethyl ketal, o-methoxybenzophenone, triphenylphosphine, tri-o-tolylphosphine, benz[a]anthracene-7,12-dione, 2,2-diethoxyacetophenone, benzyl ketals, such as benzyl dimethyl ketal, 2-methyl-1-[4-(methylthio)phenyl]-2-morpholinopropan-1-one, anthraquinones such as 2-methylanthraquinone, 2-ethylanthraquinone, 2-tert-butylanthraquinone, 1-chloroanthraquinone, 2-amylanthraquinone and 2,3-butanedione.

Also suitable are non-yellowing or low-yellowing photoinitiators of the phenylglyoxalic ester type.

Mixtures of different photoinitiators can also be used. Typical mixtures comprise, for example, 2-hydroxy-2-methyl-1 -phenylpropan-2-one and 1-hydroxycyclohexyl phenyl ketone, bis(2,6-dimethoxybenzoyl)-2,4,4-trimethylpentylphosphine oxide and 2-hydroxy-2-methyl-1-phenylpropan- 1-one, benzophenone and 1-hydroxycyclohexyl phenyl ketone, bis(2,6-dimethoxybenzoyl) -2,4,4-trimethylpentylphosphine oxide and 1-hydroxycyclohexyl phenyl ketone, 2,4,6-trimethylbenzoyldiphenylphosphine oxide and 2-hydroxy-2-methyl-1-phenylpropan- 1-one, 2,4,6-trimethylbenzophenone and 4-methylbenzophenone, or 2,4,6-trimethylbenzophenone and 4-methylbenzophenone and 2,4,6-trimethylbenzoyldiphenylphosphine oxide.

Preferred photoinitiators are:
2,4,6-trimethylbenzoyldiphenylphosphine oxide,
ethyl 2,4,6-trimethylbenzoylphenylphosphinate,
bis(2,4,6-trimethylbenzoyl)phenylphosphine oxide,
benzophenone,
1-benzoylcyclohexan-1-ol,
2-hydroxy-2,2-dimethylacetophenone, and
2,2-dimethoxy-2-phenylacetophenone.

The coating compositions comprise the photoinitiators preferably in an amount of 0.05 to 10% by weight, more preferably 0.1 to 8% by weight, more particularly 0.2 to 5% by weight, based on the total amount of the curable components present in the coating compositions.

The surfaces of solid substrates are coated with the tetrahydrofuran derivatives (I) for inventive use by customary methods known to the skilled person, wherein the desired tetrahydrofuran derivative (I), or a coating composition comprising one or more compounds (I), is applied in the desired thickness to the substrate and is at least partly radiation-cured. Complete radiation curing is preferred here. This operation may be repeated one or more times if desired. Application to the substrate may take place in a known way, as for example by spraying, troweling, knife coating, brushing, rolling, roller coating, pouring, laminating, in-mold coating or coextruding, preferably by spraying and roller coating. Spraying methods employed may for example be compressed-air, airless or electrostatic spraying methods.

As the skilled person is aware, radiation curing refers to the radical polymerization of polymerizable compounds that is induced by electromagnetic and/or particulate radiation. The use of UV light or electron beams (electron beams: 150 to 300 keV) is preferred. Especially preferred is UV light in the wavelength range from 200 to 500 nm, and more particularly from 250 to 400 nm.

The coating thickness is set preferably such that the dry film thickness is in the range from 30 to 200 μm, and preferably in the range of 50-150 μm. As the skilled person is aware, dry film thickness refers to the layer thickness of a dried or cured coating. The concept of drying includes the evaporation of solvents present in a coating composition, such as water or organic solvents, for example. The concept of curing includes the crosslinking of the coating composition. It may be especially emphasized that the concept of the dry film thickness here is to be understood, purely on a phenomenological basis, as the layer thickness possessed by a dry and/or cured coating.

Radiation curing may if desired take place at relatively high temperatures. Preferred in that case is a temperature above the T_{g} of the radiation-curable binder (T_{g}=glass transition temperature).

Radiation curing may take place under an oxygen-containing atmosphere or under inert gas, the latter being preferred.

In addition to radiation curing, there may be further curing mechanisms involved, as for example thermal, moisture, chemical and/or oxidative curing.

If desired, if there are two or more coats of the coating material applied one over another, drying and/or radiation curing may take place after each coating operation.

Examples of suitable radiation sources for the radiation curing are low-pressure mercury emitters, medium-pressure mercury emitters and high-pressure emitters, and also fluorescent tubes, pulsed emitters, metal halide lamps, lasers, pulsed lamps (flash light), halogen lamps, and electronic flash devices, by means of which radiation curing without photoinitiator is possible, or excimer emitters.

Two or more radiation sources may also be used for the radiation curing, for example, two to four. If desired, these sources may also each emit in different wavelength ranges.

Irradiation may optionally also be carried out in the absence of oxygen, such as under an inert gas atmosphere, for example. Suitable inert gases are preferably nitrogen, noble gases, carbon dioxide, or combustion gases.

A further subject of the invention in accordance with the statements made above is a method for coating the surfaces of solid substrates, by applying tetrahydrofuran derivatives (I)

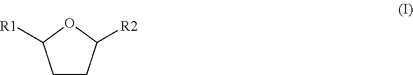

in which the radical R1 has the definition (CH₂═CH—CO—O—(CHR3—CH₂—O)ₘ—CH₂)— and the radical R2 has the definition (CH₂═CH—CO—O—(CHR4—CH₂—O)ₙ—CH₂)—, in which the radicals R3 and R4 independently of one another are hydrogen or methyl, and with the proviso that the sum of the indices m and n is a number in the range from 0 to 20, or coating compositions which comprise one or more compounds (I), to the surface of a solid substrate and subsequently carrying out radiation curing, more particularly by means of UV light.

“Coating” refers to methods which serve for the application of a firmly adhering layer to the surface of a workpiece—the substrate. The applied layer is referred to as a coating. The customary coating methods differ in the nature of the application of the coating compositions, as chemical, mechanical, thermal and thermomechanical methods. In the context of the present invention, UV curing is preferred, which induces chemical crosslinking of the compounds (I) present in the coating compositions.

### EXAMPLES

### Methods of Measurement and Testing

Viscosity: The viscosity of the substances as such was measured using a Brookfield viscometer at 25° C., shear rate of 1000 s-1, in accordance with DIN EN ISO 3219/A.3.

Pendulum damping (PD): Pendulum damping (often also referred to as pendulum hardness) of coatings resulting from application of the substances under test to the surfaces of solid substrates and their curing by UV radiation, the so-called Konig pendulum hardness, was measured according to DIN 53157. In the case of this method, the pendulum damping is reported in seconds.

Erichsen cupping (Ew): The Erichsen cupping is a measure of the elasticity of coatings. The Erichsen cupping of coatings resulting from application of the substances under test to the surfaces of solid substrates and their curing by UV radiation was measured according to DIN ISO 1520. The Erichsen cupping is reported in [mm].

Adhesion (cross-cut value=G-value): The adhesion to plastics was determined by the cross-cut method according to DIN EN ISO 2409, with the G-values being situated, according to the school-grade system, in the range from 0 to 5. Here, 0 represents the best and 5 the worst score on the scale. The plastic used for coating was Stamylan.

Iodine color number: The iodine color number was measured using the Lange Lico 400 instrument in accordance with DIN 6162

### Substances Used

TBABr: Tetrabutylammonium bromide (CAS No. 1643-19-2)

Glycidyl ether: Pentaerythritol di/tri-glycidyl ether (CAS No. 30973-88-7), “Ipox CL 16” (from Ipox)

THF-diol: 2,5-Dimethyloltetrahydrofuran; technical mixture with a molar cis/trans ratio of 90:10.

THF-diol-5.3PO: Reaction product of 7.5 mol of THF-diol with 39.7 mol of propylene oxide. Preparation took place as indicated below: 995.0 g of THF-diol and 10.0 g of solid KOH were charged to a 5 L reactor at 25° C. This reactor was then inertized with nitrogen. The reactor was heated to 120° C. and 2300 g of propylene oxide were metered in. After a reaction time of 4 hours, the reactor was evacuated under full vacuum at 50° C. for 30 minutes and then cooled to 25° C. The product was worked up by neutralization with ion exchange materials (Ambosol) and water, vacuum distillation and filtration. The product obtained was a pale liquid. 3313.4 g of product were obtained. The resulting polyether had the following characteristics:

OH number: 260 mg KOH/g
Viscosity (25° C.): 152 mPas

THF-diol-7EO: Reaction product of 7.5 mol of THF-diol with 52.3 mol of ethylene oxide. 990.0 g of THF-diol and 9.9 g of solid KOH were charged to a 5 L reactor at 25° C. This reactor was then inertized with nitrogen. The reactor was heated to 120° C. and 2300 g of ethylene oxide were metered in. After a reaction time of 4 hours, the reactor was evacuated under full vacuum at 50° C. for 30 minutes and then cooled to 25° C. The product was worked up by neutralization with ion exchange materials (Ambosol) and water, vacuum distillation and filtration. The product obtained was a pale liquid. 3240.3 g of product were obtained. The resulting polyether had the following characteristics:

OH number: 272 mg KOH/g
Viscosity (25° C.): 314 mPas

### EXAMPLES

### Example 1

Preparation of THF-diol diacrylate

115.35 g (1.75 mol of OH) THF-diol with an OH number of 424 mg KOH/g, 143.66 g of acrylic acid, 86.33 g of cyclohexane and 10.4 g of methanesulfonic acid 70% eq. were combined in the presence of a stabilizer mixture composed of 0.26 g of Kerobit, 0.78 g of methylhydroquinone, 0.27 g of hypophosphorous acid (50% eq.) and 7.8 mg of phenothiazine, the stabilizer mixture being present in solution in 1.0 g of acrylic acid, and the components were esterified at 90-95° C. After 5 hours a conversion of 90% was achieved. Then the cyclohexane and excess acrylic acid were removed under reduced pressure to an acid number (AN) of 49 mg KOH/g of substance.

Viscosity: 27 mPas
Iodine color number: 42

### Example 2

THF-diol-7EO diacrylate

In a 500 ml three-neck flask, 158.86 g (0.72 mol of OH) THF-7EO, 59.41 g of acrylic acid, 86.33 g of methylcyclohexane and 6.5 g of conc. sulfuric acid were combined in the presence of a stabilizer mixture composed of 1.0 g of Kerobit, 4.36 g of methylhydroquinone and 4.36 g of hypophosphorous acid (50% eq.) and the components were esterified at 101-105° C. Over the course of 6.5 hours, a conversion of 78% was achieved. Then the methylcyclohexane and the excess acrylic acid were removed under reduced pressure to an acid number (AN) of 53 mg KOH/g of substance. This was followed by conversion of the residual acrylic acid into 200 g of crude ester using 31.7g of Ipox CL 16, with catalysis by TBABr (4 g) at 107-108° C., until the acid number (AN) reached 4.0, followed by filtration of the product on a Seitz K300 filter. The product—a mixture of THF-diol-7EO diacrylate and the scavenging products (i.e., products of the reaction of excess acrylic acid with Ipox CL 16)—was characterized as follows:

Viscosity: 340 mPas
Iodine color number: 0.9

### Example 3

Preparation of THF-diol-5.3 PO diacrylate

In a 2000 ml three-neck flask, 884.76 g (4.0 mol of OH) THF-diol-5.3 PO, 315.23 g of acrylic acid, 400 g of methylcyclohexane and 8.1 g of methanesulfonic acid (70% eq.) were combined in the presence of a stabilizer mixture composed of 6.0 g of Kerobit, 24.0 g of methylhydroquinone and 24.0 g of hypophosphorous acid (50% eq.) and the components were esterified at 101-105° C. Over the course of 8 hours, a conversion of 79% was achieved. Then the methylcyclohexane and the excess acrylic acid were removed under reduced pressure to an acid number (AN) of 46 mg KOH/g of substance. This was followed by conversion of the residual acrylic acid using 155.3 g of Ipox CL 16, with catalysis by TBABr (25.71 g) at 107-108° C., to an AN of 3.9, followed by filtration of the product on a Seitz K300 filter. The product—a mixture of THF-diol-7PO diacrylate and the scavenging products (i.e., products of the reaction of excess acrylic acid with Ipox CL 16)—was characterized as follows:

Viscosity: 460 mPas
Iodine color number: 0.4

### Example 4

Preparation of THF-diol-5.3PO diacrylate

In a 2000 ml three-nexk flask, 879.51 g (4.0 mol of OH) THF-diol-5.3 PO, 320.49 g of acrylic acid, 228 g of cyclohexane and 75.30 g of p-toluenesulfonic acid (65% eq.) were combined in the presence of a stabilizer mixture composed of 0.86 g of a 31.5% by weight strength aqueous solution of CuCl₂, 0.24 g of methylhydroquinone and 3 g of H₃PO₂, and the components were esterified at 99° C. Over the course of 8 hours, a conversion of 79% was achieved. The product was worked up by aqueous extraction of the excess acrylic acid, removal of the solvent under reduced pressure, and subsequent filtration of the product on a Seitz K300 filter. The product was characterized as follows:

Viscosity: 70 mPas
Iodine color number: 5.2

### APPLICATION EXAMPLES

In the application examples below, the coating compositions were cured using an IST UV system (system type: M-40-2x1-R-TR-SLC-SO-inert; lamp 1: IST UV lamp M400 U2HC; lamp 2: IST UV lamp M400 U2H)

### Application Example 1

### Determination of the Film Properties of the Product of Example 1

The product of Example 1 was admixed with 5% by weight—based on this product—of the photoinitiator Irgacure 500. The coating composition thus prepared was applied using a four-way bar applicator to Stamylan, the slot width of the bar coater being 200 μm (hence implying that the wet film thickness of the applied coating was 200 μm). Curing took place under a nitrogen atmosphere with an energy input of 1900 mJ/cm². This was followed by determination of pendulum damping (PD), Erichsen cupping (Ew) and cross-cut value. The results obtained were as follows:

### PD=113 s

### Ew=1.6 mm

Cross-cut value=0

### Application Example 2

### Determination of the Film Properties of the Product of Example 2

The product of Example 2 was admixed with 5% by weight—based on this product—of the photoinitiator Irgacure 500. The coating composition thus prepared was applied using a four-way bar applicator to Stamylan, the slot width of the bar coater being 200 μm (hence implying that the wet film thickness of the applied coating was 200 μm). Curing took place under a nitrogen atmosphere with an energy input of 1900 mJ/cm². This was followed by determination of pendulum damping (PD), Erichsen cupping (Ew) and cross-cut value. The results obtained were as follows:

### PD=88 s

### Ew=4.1 mm

Cross-cut value=1

### Application Example 3

### Determination of the Film Properties of the Product of Example 3

The product of Example 3 was admixed with 5% by weight—based on this product—of the photoinitiator Irgacure 500. The coating composition thus prepared was applied using a four-way bar applicator to Stamylan, the slot width of the bar coater being 100 μm (hence implying that the wet film thickness of the applied coating was 100 μm). Curing took place under a nitrogen atmosphere with an energy input of 1900 mJ/cm². This was followed by determination of pendulum damping (PD), Erichsen cupping (Ew) and cross-cut value. The results obtained were as follows:

### PD=123 s

### Ew=3.3 mm

Cross-cut value=1

### Application Example 4

### Determination of the Film Properties of the Product of Example 4

The product of Example 4 was admixed with 5% by weight—based on this product—of the photoinitiator Irgacure 500. The coating composition thus prepared was applied using a four-way bar applicator to Stamylan, the slot width of the bar coater being 100 μm (hence implying that the wet film thickness of the applied coating was 100 μm). Curing took place under a nitrogen atmosphere with an energy input of 1900 mJ/cm². Addition of 5% Irgacure 500 as photoinitiator; application with four-way applicators to Stamylan; slot width of bar coater 100 μm (thus implying that the wet film thickness of the applied coating is 100 μm); curing under nitrogen atmosphere with an energy input of 1900 mJ/cm². This was followed by determination of pendulum damping (PD), Erichsen cupping (Ew) and cross-cut value. The results obtained were as follows:

### PD=141 s

### Ew=3.1 mm

Cross-cut value=0

1. Tetrahydrofuran derivatives of the formula (I) 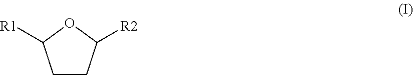
in which radical R1 has the definition (CH₂═CH—CO—O—(CHR3—CH₂—O)ₘ—CH₂)— and radical R2 has the definition (CH₂═CH—CO—O—(CHR4—CH₂—O)ₙ—CH₂)—, in which radicals R3 and R4 independently of one another are hydrogen or methyl, with the proviso that the sum of the indices m and n is a number in the range from 0 to 20.

2. The tetrahydrofuran derivatives according to claim 1, with the proviso that the sum of the indices m and n is a number in the range from 5 to 12.

3. A coating composition comprising one or more tetrahydrofuran derivatives of the formula (I) 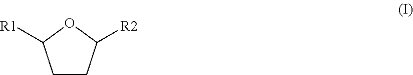
in which radical R1 has the definition (CH₂═CH—CO—O—(CHR3—CH₂—O)ₘ—CH₂)— and radical R2 has the definition (CH₂═CH—CO—O—(CHR4—CH₂—O)ₙ,—CH₂)—, in which radicals R3 and R4 independently of one another are hydrogen or methyl, with the proviso that the sum of the indices m and n is a number in the range from 0 to 20.

4. The coating composition according to claim 3, with the proviso that the sum of the indices m and n is a number in the range from 5 to 12.

5. (canceled)

6. (canceled)

7. (canceled)

8. (canceled)

9. (canceled)

10. (canceled)

11. A method for coating the surfaces of solid substrates, comprising applying a tetrahydrofuran derivative (I) 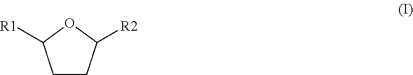
in which radical R1 has the definition (CH₂═CH—CO—O—(CHR3—CH₂—O)ₘ,—CH₂)—and radical R2 has the definition (CH₂═CH—CO—O—(CHR4—CH₂—O)ₙ,—CH₂)—, in which radicals R3 and R4 independently of one another are hydrogen or methyl, with the proviso that the sum of the indices m and n is a number in the range from 0 to 20, or coating compositions which comprise one or more compounds (I), to the surface of a solid substrate and carrying out radiation curing.

12. The method according to claim 11, wherein the solid substrate is a plastic.

13. The method according to claim 11, wherein the radiation curing is curing with UV light of a wavelength in the range from 200 to 500 nm.

14. The method according to claim 11, wherein the solid substrate is a plastic and the radiation curing is curing with UV light of a wavelength in the range from 250 to 400 nm.
